(19) United States
(12) Patent Application Publication
(10) Pub. No.: US 2026/0193607 A1
(43) Pub. Date: Jul. 9, 2026

(54) HIGH-THROUGHPUT MANUFACTURING AND SCREENING OF CAR-T CELLS

(71) Applicant: Kite Pharma, Inc., Santa Monica, CA (US)

(72) Inventors: Alessandro P. Calo, Santa Monica, CA (US); Nicole M.W. Cannon, San Diego, CA (US); Sean C. Yoder, Simi Valley, CA (US)

(21) Appl. No.: 19/422,103

(22) Filed: Dec. 16, 2025

Related U.S. Application Data
(60) Provisional application No. 63/735,453, filed on Dec. 18, 2024.

(51) Int. Cl. C12N 5/0783; C12N 15/86; G01N 33/50

(52) U.S. Cl. CPC C12N 5/0636; C12N 15/86; G01N 33/5011; C12N 2510/00; C12N 2740/15043

(57) ABSTRACT

Described here are automatable high-throughput processes for manufacturing and screening engineered T cells, such as chimeric antigen receptor (CAR)-T cells and TCR-T cells.

### CROSS-REFERENCE TO RELATED APPLICATIONS

The present application claims the priority benefit of U.S. Provisional Application No. 63/735,453, filed Dec. 18, 2024, which is hereby incorporated by reference in its entirety.

### BACKGROUND

Immune cells can be modified to target and kill cancer cells. Chimeric antigen receptors (CARs) and engineered T cell receptors (TCRs), which include binding domains capable of interacting with a particular tumor antigen, allow the immune cells to target and kill cancer cells that express the particular tumor antigen.

A significant challenge comes with the highly complicated cell engineering and production process, which typically takes at least a week, and can take as long as several weeks. During the process, lymphocytes collected from the patients must be shipped to the process center, while the produced cells have to be cryopreserved and then shipped back to the patient for implantation. This highly complicated process necessarily leads to high costs, and limited clinical applications.

CAR architectures can have significant influence on the function of a CAR-T cell. A CAR architecture suitable for a particular target or disease, therefore, needs to be carefully designed and selected, preferably from large and architecturally diverse CAR libraries. The lengthy and complicated production process, however, presents a significant challenge for CAR-T screening. There is a strong need to efficiently screen for CAR-T at a large scale.

### SUMMARY

The instant disclosure describes a new, automated, high-throughput process for the manufacturing and screening of CAR-T cells. In one embodiment, a method is provided for identifying T cells for expressing proteins encoded in polynucleotide vectors, comprising: adding, to each of a plurality of T cell samples, one of a plurality (N) of polynucleotide vectors at one of a plurality (M) of amounts, each encoding a protein; incubating each sample to allow the T cells to be transduced or transfected with the vector and express the protein; measuring, in each sample, an expression level of the protein; identifying, for each vector, a selected sample with one of the M amounts of the vector added, wherein the identification is at least in part based on the expression level; and culturing each of the selected samples, thereby producing T cells expressing the proteins. In some embodiments, N is at least 2; M is at least 2; and for each vector, the difference between the highest amount and the lowest amount within the M amounts is at least 10 fold.

In some embodiments, each of the proteins is a chimeric antigen receptor (CAR) or a T cell receptor (TCR). In some embodiments, the T cells are primary T cells.

In some embodiments, each of the polynucleotide vectors is a viral or retroviral vector. In some embodiments, the retroviral vector is a lentiviral vector.

In some embodiments, the expression level is a protein positivity level defined as a proportion of T cells of total live T cells in the sample that express the protein. In some embodiments, identification of the selected samples comprises comparing the expression levels from each of the M amounts to predetermined expression level cutoff values. In some embodiments, the predetermined expression level cutoff values comprise a maximum cutoff, a minimum cutoff and a goal cutoff.

In some embodiments, for each vector:
(a) if the protein positivity levels of all M amounts are greater than the maximum cutoff, then the sample with the lowest protein positivity level is identified as the selected sample;
(b) if the protein positivity levels of all M amounts are lower than the minimum cutoff, then the sample with the highest protein positivity level is identified as the selected sample;
(c) if at least two of the protein positivity levels of all M amounts are between the maximum cutoff and the minimum cutoff, then the sample with a protein positivity level closest to the goal cutoff is identified as the selected sample; or
(d) if only one of the protein positivity levels of all M amounts is between the maximum cutoff and the minimum cutoff, then the sample having the protein positivity level between the maximum cutoff and the minimum cutoff is identified as the selected sample.

In some embodiments, M is at least 3. In some embodiments, M is 4.

In some embodiments, for each vector, the difference between the highest amount and the lowest amount within the M amounts is at least 20 fold. In some embodiments, for each vector, the difference between the highest amount and the lowest amount within the M amounts is at least 40 fold.

In some embodiments, all samples not identified as selected samples are not cultured together with the selected samples.

In some embodiments, the T cells are selected from a plurality of donors by a method comprising:
contacting T cells from each donor with a target tumor cell and measuring a first cytotoxicity level;
contacting T cells from the same donor, which have been engineered to express a reference CAR targeting a tumor marker associated with the target tumor cell, with the target tumor cell and measuring a second cytotoxicity level; and
selecting T cells, among the plurality of donors, having the highest difference between the second cytotoxicity level and the first cytotoxicity level.

In some embodiments, the highest difference is the highest fold difference.

In some embodiments, N is at least 8. In some embodiments, N is at least 32.

In some embodiments, the method further comprises activating the T cells with an anti-CD3 and/or anti-CD28 antibody.

In some embodiments, the method further comprises normalizing the number of T cells among the T cell samples.

In some embodiments, the M amounts of the vectors are made by dilutions from an initial amount. In some embodiments, the initial amounts are normalized among the vectors. In some embodiments, the initial amounts are not normalized among the vectors.

In some embodiments, the method further comprises measuring the cytotoxicity of each cultured sample.

### BRIEF DESCRIPTION OF THE DRAWINGS

FIG. 1 is a graph showing transduction efficiencies of primary T cells from donors and Jurkat cell lines according to an embodiment of the disclosure.

FIG. 2 is two graphs comparing the cytotoxicity of T cells with or without a transduced CAR after 1 day (left graph) and after 4 days (right graph), according to an embodiment of the disclosure.

### DETAILED DESCRIPTION

### Definitions

In order for the present disclosure to be more readily understood, certain terms are first defined below. Additional definitions for the following terms and other terms are set forth throughout the Specification.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless specifically stated or evident from context the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within one or more than one standard deviation per the practice in the art. “About” or “comprising essentially of” can mean a range of up to 10% (i.e., +10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “comprising essentially of” should be assumed to be within an acceptable error range for that particular value or composition.

“Administering” refers to the physical introduction of an agent to a subject, such as a modified T cell disclosed herein, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The term “allogeneic” refers to any material derived from one individual which is then introduced to another individual of the same species, e.g., allogeneic T cell transplantation.

The term “antibody” (Ab) includes, without limitation, a glycoprotein immunoglobulin which binds specifically to an antigen. In general, and antibody can comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system. In general, human antibodies are approximately 150 kD tetrameric agents composed of two identical heavy (H) chain polypeptides (about 50 kD each) and two identical light (L) chain polypeptides (about 25 kD each) that associate with each other into what is commonly referred to as a “Y-shaped” structure. The heavy and light chains are linked or connected to one another by a single disulfide bond; two other disulfide bonds connect the heavy chain hinge regions to one another, so that the dimers are connected to one another and the tetramer is formed. Naturally-produced antibodies are also glycosylated, e.g., on the CH2 domain.

An “antigen binding molecule,” “antigen binding portion,” “antigen binding fragment,” or “antibody fragment” refers to any molecule that comprises the antigen binding parts (e.g., CDRs) of the antibody from which the molecule is derived. An antigen binding molecule can include the antigenic complementarity determining regions (CDRs). Examples of antibody fragments include, but are not limited to, Fab, Fab′, F(ab′) 2, and Fv fragments, dAb, linear antibodies, scFv antibodies, and multispecific antibodies formed from antigen binding molecules. Peptibodies (i.e., Fc fusion molecules comprising peptide binding domains) are another example of suitable antigen binding molecule. In some embodiments, the antigen binding molecule binds to an antigen on a tumor cell. In some embodiments, the antigen binding molecule binds to an antigen on a cell involved in a hyperproliferative disease or to a viral or bacterial antigen. In certain embodiments an antigen binding molecule is a chimeric antigen receptor (CAR) or an engineered T cell receptor (TCR).

The term “variable region” or “variable domain” is used interchangeably. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody or an antigen-binding molecule thereof.

The terms “VH” and “VH domain” are used interchangeably to refer to the heavy chain variable region of an antibody or an antigen-binding molecule thereof.

A number of definitions of the CDRs are commonly in use: Kabat numbering, Chothia numbering, AbM numbering, or contact numbering. The AbM definition is a compromise between the two used by Oxford Molecular's AbM antibody modelling software. The contact definition is based on an analysis of the available complex crystal structures.

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient.

“Chimeric antigen receptor” or “CAR” refers to a molecule engineered to comprise a binding motif and a means of activating immune cells (for example T cells such as naive T cells, central memory T cells, effector memory T cells or combination thereof) upon antigen binding. CARs are also known as artificial T cell receptors, chimeric T cell receptors or chimeric immunoreceptors. In some embodiments, a CAR comprises a binding motif, an extracellular domain, a transmembrane domain, one or more co-stimulatory domains, and an intracellular signaling domain. A T cell that has been genetically engineered to express a chimeric antigen receptor may be referred to as a CAR T cell. “Extracellular domain” (or “ECD”) refers to a portion of a polypeptide that, when the polypeptide is present in a cell membrane, is understood to reside outside of the cell membrane, in the extracellular space.

A “T cell receptor” or “TCR” refers to antigen-recognition molecules present on the surface of T cells. During normal T cell development, each of the four TCR genes, a, B, Y, and 8, may rearrange leading to highly diverse TCR proteins.

The term “heterologous” means from any source other than naturally occurring sequences. For example, a heterologous sequence included as a part of a costimulatory protein is amino acids that do not naturally occur as, i.e., do not align with, the wild type human costimulatory protein. For example, a heterologous nucleotide sequence refers to a nucleotide sequence other than that of the wild type human costimulatory protein-encoding sequence.

The immune cells of the immunotherapy can come from any source known in the art. For example, immune cells can be differentiated in vitro from a hematopoietic stem cell population, or immune cells can be obtained from a subject. Immune cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the immune cells can be derived from one or more immune cell lines available in the art. Immune cells can also be obtained from a unit of blood collected from a subject using any number of techniques known to the skilled artisan, such as FICOLL™ separation and/or apheresis. Additional methods of isolating immune cells for an immune cell therapy are disclosed in U.S. Patent Publication No. 2013/0287748, which is herein incorporated by references in its entirety.

A “patient” includes any human who is afflicted with a cancer (e.g., a lymphoma or a leukemia). The terms “subject” and “patient” are used interchangeably herein.

The term “pharmaceutically acceptable” refers to a molecule or composition that, when administered to a recipient, is not deleterious to the recipient thereof, or that any deleterious effect is outweighed by a benefit to the recipient thereof. With respect to a carrier, diluent, or excipient used to formulate a composition as disclosed herein, a pharmaceutically acceptable carrier, diluent, or excipient must be compatible with the other ingredients of the composition and not deleterious to the recipient thereof, or any deleterious effect must be outweighed by a benefit to the recipient. The term “pharmaceutically acceptable carrier” means a pharmaceutically-acceptable material, composition or vehicle, such as a liquid or solid filler, diluent, excipient, or solvent encapsulating material, involved in carrying or transporting an agent from one portion of the body to another (e.g., from one organ to another). Each carrier present in a pharmaceutical composition must be “acceptable” in the sense of being compatible with the other ingredients of the formulation and not deleterious to the patient, or any deleterious effect must be outweighed by a benefit to the recipient. Some examples of materials which may serve as pharmaceutically acceptable carriers comprise: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

The term “pharmaceutical composition” refers to a composition in which an active agent is formulated together with one or more pharmaceutically acceptable carriers. In some embodiments, the active agent is present in a unit dose amount appropriate for administration in a therapeutic regimen that shows a statistically significant probability of achieving a predetermined therapeutic effect when administered to a relevant subject or population. In some embodiments, a pharmaceutical composition may be formulated for administration in solid or liquid form, comprising, without limitation, a form adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions.

The term “reference” describes a standard or control relative to which a comparison is performed. For example, in some embodiments, an agent, animal, individual, population, sample, sequence, or value of interest is compared with a reference or control that is an agent, animal, individual, population, sample, sequence, or value. In some embodiments, a reference or control is tested, measured, and/or determined substantially simultaneously with the testing, measuring, or determination of interest. In some embodiments, a reference or control is a historical reference or control, optionally embodied in a tangible medium. Generally, a reference or control is determined or characterized under comparable conditions or circumstances to those under assessment. When sufficient similarities are present to justify reliance on and/or comparison to a selected reference or control.

A “therapeutically effective amount,” “effective dose,” “effective amount,” or “therapeutically effective dosage” of a therapeutic agent, e.g., engineered CAR T cells, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The terms “transduction” and “transduced” refer to the process whereby foreign DNA is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In one embodiment, “treatment” or “treating” includes a partial remission. In another embodiment, “treatment” or “treating” includes a complete remission. In some embodiments, treatment may be of a subject who does not exhibit signs of the relevant disease, disorder and/or condition and/or of a subject who exhibits only early signs of the disease, disorder, and/or condition. In some embodiments, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition.

The term “vector” refers to a recipient nucleic acid molecule modified to comprise or incorporate a provided nucleic acid sequence. One type of vector is a “plasmid,” which refers to a circular double stranded DNA molecule into which additional DNA may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) may be integrated into the genome of a host cell upon introduction into the host cell, and thereby are replicated along with the host genome. Moreover, certain vectors comprise sequences that direct expression of inserted genes to which they are operatively linked. Such vectors may be referred to herein as “expression vectors.” Standard techniques may be used for engineering of vectors, e.g., as found in Sambrook et al., Molecular Cloning: A Laboratory Manual (2d ed., Cold Spring Harbor Laboratory Press, Cold Spring Harbor, N.Y. (1989)), which is incorporated herein by reference.

### High-Throughput CAR-T Screening

The conventional autologous CAR cell manufacturing process takes many days and is highly complicated. Meanwhile, the CAR architectures can have significant influence on the function of a CAR cell. Designing and identifying a suitable CAR architecture, therefore, can greatly improve the efficiency of CAR cell manufacturing and clinical use.

Conventionally, the transduction and expression efficiencies of a CAR is tested in cell lines, instead of primary donor T cells. Cell lines, such as Jurkat cells, are more readily available, more reproducible across labs, and less expensive. CARs with the best performance in Jurkat cells are selected for CAR cell manufacturing.

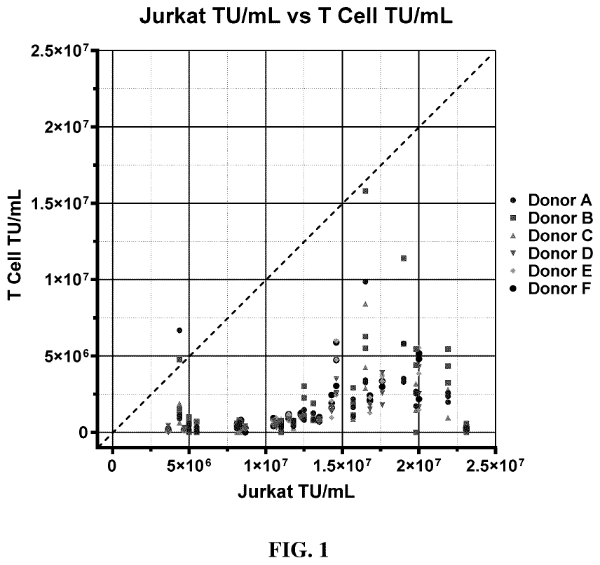

In a first unexpected discovery, however, the instant inventors have demonstrated that there was a lack of correlation between CAR lentiviral (LVV) titers in Jurkat cells and LVV titers in donor T cells (Example 1 and FIG. 1). Therefore, it is contemplated that CAR screening should be carried out in primary donor T cells, instead of established cell lines, such as Jurkat cells.

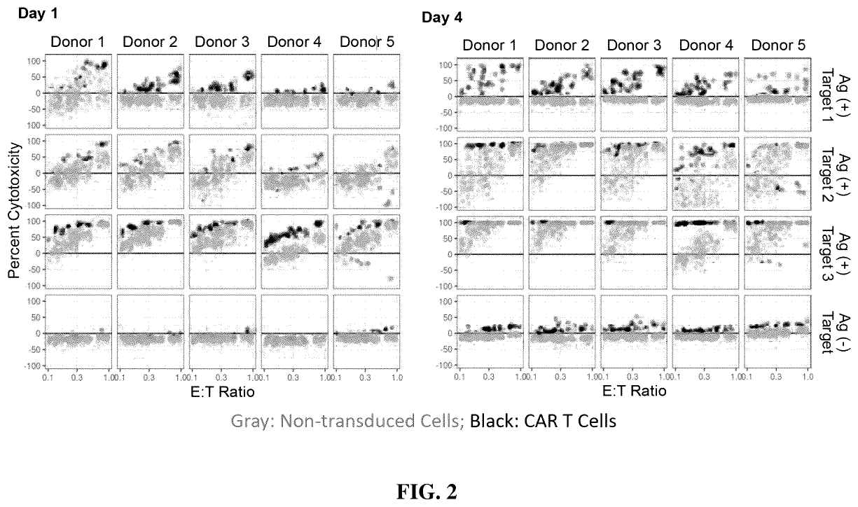

Another unexpected finding was that, even among primary donor T cells or T cell batches, there can be considerable differences with respect to their ability to test potential CAR-T efficacy. As shown in FIG. 2, some donor T cells, even without being transduced with a CAR, exhibited high background cytotoxicity (noise). Such high background noise, therefore, can confound the measurement of CAR efficacy. Accordingly, in some embodiments, donor T cells are screened before they are used for CAR screening. In some embodiments, donor T cells with relatively higher signal/noise ratios are selected, wherein “signal” may be measured as cytotoxicity with a candidate CAR, and “noise” may be measured as cytotoxicity without the CAR.

Preferably with such considerations, a suitable high-throughput CAR screening process can be established. Such a process was tested in Example 1, which observed that the screening was able to reduce the CAR-T drop out rates from about 60% to about 14%. One important element of the screening process is that, for each protein-encoding vector (e.g., a lentiviral vector encoding a CAR), at least two different amounts of the vectors are tested and the one that is potentially optimal for the downstream process is selected.

In a preferred embodiment, these different amounts are made by having an initial solution containing the vector, with one or more dilutions. For instant, in Example 1, four different dilutions (1×, 10×, 20× and 40×) were made and tested for the LVV. There, the difference between the highest amount and the lowest amount is 40×. In some embodiments, the highest difference may as smaller (e.g., 2×, 5×, 10× or 20×), or higher (e.g., 50×, 60×, 80×, 100× or 200×), without limitation. In some embodiments, each vector can be provided at 2, 3, 4, 5, 6, 7, 8 or more different amounts.

In accordance with one embodiment of the present disclosure, therefore, provided is a method for identifying T cells for expressing proteins encoded in polynucleotide vectors, wherein the T cells are transduced or transfected with the vectors. In one embodiment, the method entails
(a) adding, to each of a plurality of T cell samples, one of a plurality (N) of polynucleotide vectors at one of a plurality (M) of amounts, each encoding a protein;
(b) incubating each sample to allow the T cells to be transduced or transfected with the vector and express the protein;
(c) measuring, in each sample, an expression level of the protein;
(d) identifying, for each vector, a selected sample with one of the M amounts of the vector added, wherein the identification is at least in part based on the expression level; and
(e) culturing each of the selected samples, thereby producing T cells expressing the proteins.

Each such high-throughput testing, therefore, can include N different vectors each having M different amount. For instance, as tested in Example 1, N was 96 and M was 4 (1×, 10×, 20× and 40×), and thus all 384 samples could be accommodated in a 384-well plate, readily handled with liquid handling.

In some embodiments, N is at least 2, or at least 4, 8, 16, 32, 48, 64, 96, without limitation. In some embodiments, M is at least 2, 3, 4, 6 or 8. In some embodiments, Mis 2. In some embodiments, M is 4. In some embodiments, Mis 8.

In some embodiments, for each vector, the difference between the highest amount and the lowest amount within the M amounts is at least 5 fold. In some embodiments, for each vector, the difference between the highest amount and the lowest amount within the M amounts is at least 10 fold. In some embodiments, for each vector, the difference between the highest amount and the lowest amount within the M amounts is at least 20 fold. In some embodiments, for each vector, the difference between the highest amount and the lowest amount within the M amounts is at least 30 fold. In some embodiments, for each vector, the difference between the highest amount and the lowest amount within the M amounts is at least 40 fold.

The protein encoded by each vector, in some embodiments, is a receptor that can be expressed on T cell surface. In one embodiment, it is a chimeric antigen receptor (CAR). In another embodiment, it is a T cell receptor (TCR).

As described above, it was discovered herein that established cell lines (e.g., Jurkat cell) may not provide a good evaluation platform for autologous therapies. Accordingly, in some embodiments, the T cells used in the present technology are primary T cells.

### Vectors and Transduction

In some embodiments, the vectors can be viral vectors, such as lentiviral vectors, as well as retroviral vectors. Several recombinant viruses have been used as viral vectors to deliver genetic material to a cell. Viral vectors that may be used in accordance with the transduction step may be any ecotropic or amphotropic viral vector including, but not limited to, recombinant retroviral vectors, recombinant lentiviral vectors, recombinant adenoviral vectors, and recombinant adeno-associated viral (AAV) vectors. In one embodiment, the viral vector is an MSGV1 gamma retroviral vector. In some embodiments, the vectors are non-viral vectors.

The incubation (or transduction) step can be carried out in a suitable culturing system, in particular a closed culturing system, without limitation. In certain embodiments, the closed system is a closed bag culture system, using any suitable cell culture bags (e.g., Mitenyi Biotec MACS® GMP Cell Differentiation Bags, Origen Biomedical PermaLife™ Cell Culture bags). In some embodiments, the closed system has an inner surface area of at least 500 cm². In some embodiments, the closed system has an inner surface area of at least 1000 cm², 1200 cm², 1400 cm², 1500 cm², 1600 cm², 1800 cm², 2000 cm², 2200 cm², 2500 cm², or 3000 cm². In some embodiments, the closed system has an inner surface area of not greater than 1500 cm², 1600 cm², 1800 cm², 2000 cm², 2200 cm², 2500 cm², or 3000 cm².

In some embodiments, the cell culture bags used in the closed system are coated with a recombinant human fibronectin protein. The recombinant human fibronectin fragment may include three functional domains: a central cell-binding domain, heparin-binding domain II, and a CS1-sequence. The recombinant human fibronectin protein or fragment thereof may be used to increase gene efficiency of retroviral transduction of immune cells by aiding co-localization of target cells or the vector. In certain embodiments, the recombinant human fibronectin fragment is RetroNectin® (Takara Bio, Japan). In certain embodiments, the cell culture bags may be coated with recombinant human fibronectin fragment at a concentration of about 0.1-60 μg/mL, preferably 0.5-40 μg/mL. In certain embodiments, the cell culture bags may be coated with recombinant human fibronectin fragment at a concentration of about 0.5-20 μg/mL, 20-40 μg/mL, or 40-60 μg/mL. In certain embodiments, the cell culture bags may be coated with about 0.5 μg/mL, 1 μg/mL, about 2 μg/mL, about 3 μg/mL, about 4 μg/mL, about 5 μg/mL, about 6 μg/mL, about 7 μg/mL, about 8 μg/mL, about 9 μg/mL, about 10 μg/mL, about 11 μg/mL, about 12 μg/mL, about 13 μg/mL, about 14 μg/mL, about 15 μg/mL, about 16 μg/mL, about 17 μg/mL, about 18 μg/mL, about 19 μg/mL, or about 20 μg/mL recombinant human fibronectin fragment. In certain embodiments, the cell culture bags may be coated with about 2-5 μg/mL, about 2-10 μg/mL, about 2-20 μg/mL, about 2-25 μg/mL, about 2-30 μg/mL, about 2-35 μg/mL, about 2-40 μg/mL, about 2-50 μg/mL, or about 2-60 μg/mL recombinant human fibronectin fragment. In certain embodiments, the cell culture bags may be coated with at least about 2 μg/mL, at least about 5 μg/mL, at least about 10 μg/mL, at least about 15 μg/mL, at least about 20 μg/mL, at least about 25 μg/mL, at least about 30 μg/mL, at least about 40 μg/mL, at least about 50 μg/mL, or at least about 60 μg/mL recombinant human fibronectin fragment. In certain embodiments, the cell culture bags may be coated with at least about 10 μg/mL recombinant human fibronectin fragment.

In certain embodiments, the cell culture bags used in the closed bag culture system may be blocked with human albumin serum (HSA). In an alternative embodiment, the cell culture bags are not blocked with HSA.

Once the closed system is coated with the recombinant fibronectin, a solution that includes the vector is added to the closed system so that the vector can be immobilized by the recombinant fibronectin, on the inner surface of the closed system. Such immobilization can improve the transduction efficiency once the cells are added.

In some embodiments, a total volume of at least 100 mL of the solution that contains the vector is used. In some embodiments, a total volume of no more than 500 mL of the solution that contains the vector is used.

Once the closed system is coated with the recombinant fibronectin and has immobilized the vector, the vector solution can be removed. In some embodiments, the removal of the vector solution is done by gravity drain, which helps to retain the immobilized vector on the inner surface.

T cell transduction can be carried in the coated closed system with the immobilized vectors. In some embodiments, the transduction is performed with a sample that contained the T cells. In some embodiments, the sample includes at least 2.5×10⁸ T cells. In some embodiments, the sample includes no more than 8×10⁸, 9×10⁸, or 10×10⁸ T cells.

The T cells used in the presently disclosed methods are typically obtained from a donor subject, which may be a cancer patient that is to be treated with a population of cells generated by the methods described herein (i.e., an autologous donor), or may be an individual that donates a lymphocyte sample that, upon generation of the population of cells generated by the methods described herein, will be used to treat a different individual or cancer patient (i.e., an allogeneic donor). The lymphocytes may be obtained from the donor subject by any suitable method used in the art. For example, the lymphocytes may be obtained by any suitable extracorporeal method, venipuncture, or other blood collection method by which a sample of blood and/or lymphocytes is obtained. In one embodiment, the lymphocytes are obtained by apheresis.

Optionally, in some embodiments, the method described herein further includes a step of enriching a population of T cells obtained from the donor subject, prior to the transduction. Enrichment of lymphocytes may be accomplished by any suitable separation method including, but not limited to, the use of a separation medium (e.g., Ficoll-Paque™, RosetteSep™ HLA Total Lymphocyte enrichment cocktail, Lymphocyte Separation Medium (LSA) (MP Biomedical Cat. No. 0850494X), or the like), cell size, shape or density separation by filtration or elutriation, immunomagnetic separation (e.g., magnetic-activated cell sorting system, MACS), fluorescent separation (e.g., fluorescence activated cell sorting system, FACS), or bead based column separation.

Also optionally, the methods described herein further includes a step of stimulating the T cells with one or more T cell stimulating agents. In some embodiments, the stimulation is performed prior to the transduction step. In some embodiments, the stimulation is performed after the transduction step.

Any combination of one or more suitable T cell stimulating agents may be used to stimulate (activate) the T cells. Non-limiting examples include an antibody or functional fragment thereof which targets a T-cell stimulatory or co-stimulatory molecule (e.g., anti-CD2 antibody, anti-CD3 antibody, anti-CD28 antibody, or functional fragments thereof) a T cell cytokine (e.g., any isolated, wildtype, or recombinant cytokines such as: interleukin 1 (IL-1), interleukin 2, (IL-2), interleukin 4 (IL-4), interleukin 5 (IL-5), interleukin 7 (IL-7), interleukin 15 (IL-15), tumor necrosis factor α (TNFα)), or any other suitable mitogen (e.g., tetradecanoyl phorbol acetate (TPA), phytohaemagglutinin (PHA), concanavalin A (conA), lipopolysaccharide (LPS), pokeweed mitogen (PWM)) or natural ligand to a T-cell stimulatory or co-stimulatory molecule. In some embodiments, the stimulating agent is an anti-CD3 antibody and an anti-CD28 antibody.

In some embodiments, the step of stimulating T cells as described herein may entail stimulating the T cells with one or more stimulating agents at a predetermined temperature, for a predetermined amount of time, and/or in the presence of a predetermined level of CO₂. In certain embodiments, the predetermined temperature for stimulation may be about 34° C., about 35° C., about 36° C., about 37° C., about 38° C., or about 39° C.

In some embodiments, an anti-CD3 antibody (or functional fragment thereof), an anti-CD28 antibody (or functional fragment thereof), or a combination of anti-CD3 and anti-CD28 antibodies may be used in accordance with the step of stimulating the population of T cells. Any soluble or immobilized anti-CD3 and/or anti-CD28 antibody or functional fragment thereof may be used (e.g., clone OKT3 (anti-CD3), clone 145-2C11 (anti-CD3), clone UCHT1 (anti-CD3), clone L293 (anti-CD28), clone 15E8 (anti-CD28)). In some aspects, the antibodies may be purchased commercially from vendors known in the art including, but not limited to, Miltenyi Biotec, BD Biosciences (e.g., MACS GMP CD3 pure 1 mg/mL, Part No. 170-076-116), and eBioscience, Inc. Further, one skilled in the art would understand how to produce an anti-CD3 and/or anti-CD28 antibody by standard methods. Any antibody used in the methods described herein should be produced under Good Manufacturing Practices (GMP) to conform to relevant agency guidelines for biologic products.

Culturing of transduced T cells can be done in media and conditions known in the art. In some embodiments, the culturing of the transduced T cells may be performed at a temperature and/or in the presence of CO₂. In certain embodiments, the temperature may be about 34° C., about 35° C., about 36° C., about 37° C., about 38° C., or about 39° C.

Any suitable culture medium T cell growth media may be used for culturing the cells in suspension. For example a T cell growth media may include, but is not limited to, a sterile, low glucose solution that includes a suitable amount of buffer, magnesium, calcium, sodium pyruvate, and sodium bicarbonate. In one embodiment, the culturing media is OpTmizer™ (Life Technologies), but one skilled in the art would understand how to generate similar media.

### Donor Cell Screening

Optionally, in some embodiments, before the donor T cells are used in transduction, they are screened and selected. In some embodiments, donor T cells with relatively higher signal/noise ratios are selected, wherein “signal” may be measured as cytotoxicity with a candidate CAR, and “noise” may be measured as cytotoxicity without the CAR.

In an example embodiment, the donor T cell selection method entails contacting T cells from each donor with a target tumor cell (non-transduced T cells) and measuring a first cytotoxicity level (a “noise” level), contacting T cells from the same donor, which have been engineered to express a reference CAR targeting a tumor marker associated with the target tumor cell (CAR-T cells), with the target tumor cell and measure a second cytotoxicity level (a “signal” level); and selecting T cells, among the plurality of donors, having the highest difference between the second cytotoxicity level and the first cytotoxicity level (signal to noise difference).

In some embodiments, the highest difference is the highest fold difference (e.g., highest signal/nose ratio). In some embodiments, T cells from multiple donors having the highest signal to noise differences are selected. In some embodiments, T cells from multiple donors having the lowest signal to noise differences are removed. In some embodiments, the selection is further based on other criteria, such as growth rate or transduction efficiency of the cells.

### Positivity Measurement and Sample Selection

For each sample, the protein expression is measured which is subsequently used for sample selection. In some embodiments, the protein expression level is the total amount of the protein expressed in the sample. This level may be measured, for instance, by a labeled antibody specific to the protein.

In a preferred embodiment, the protein expression level is a protein positivity level defined as a proportion of T cells of total live T cells in the sample that express the protein. For instance, if the sample includes 1,00,000 T cells, out of which 800,000 T cells are live, and 400,000 of the live cells express the protein, then the protein positivity level is 400,000/800,000=50%. Determining the number of protein-expressing T cells may be carried out with methods known in art, such as fluorescence-activated cell sorting (FACS).

Selection of a sample, from multiple samples having the same CAR but at different dilution level, for further testing or development can be done with predetermined criteria. In one example, the sample with the highest protein expression level is selected. In another example, the sample with a protein expression level closest to the medium is selected. In yet another embodiment, a set of cutoff protein expression levels are used for such selection.

An example set of selection criteria was tested in Example 1, which includes a minimum CAR positivity (“Min”), a maximum CAR positivity (“Max”) and a goal CAR positivity (“Goal”) that is between the Min and the Max. An example set of selection criteria are illustrated in Table 2. Briefly, if the CAR positivity (Pos %) of all samples>Max, then select CAR-T with lowest Pos %; if all Pos %<Min, then select CAR-T with highest Pos %; if at least two Pos % between Min and Max, then select CAR-T with Pos % closest to Goal; and if only one Pos % between Min and Max, then select this one.

Application of these cutoff values in selecting a suitable sample is illustrated in the example below. Table A lists the CAR positivity measurement results for two CAR constructs, CAR_1 and CAR_2, each had 4 different levels of dilutions. For CAR_1, the CAR positivity percentages for the four dilutions are 90.7%, 49.7%, 28.1% and 15.7%, respectively. For CAR_2, the CAR positivity percentages for the four dilutions are 92.8%, 82.7%, 60.1% and 52.3%, respectively.

The predetermined Min is 50%, Max is 75% and Goal is 60%. For CAR_1, only the 10× dilution sample has a CAR positivity % that falls between the Min and Max, and thus that sample is selected. For CAR_2, both the 20× dilution sample and the 40× dilution sample have CAR positivity % that fall between the Min and Max. Between them, the 20× dilution sample has a CAR positivity % (60.1%) that is closer to the Goal (60%), and thus it is selected.

In both examples, the CAR positivity % closest to the Goal is selected. However, it would be readily appreciated by the skilled artisan that this may not always be the case under the selection criteria of Table 2. For instance, if the four CAR positivity % are 10%, 48%, 72% and 90%, then the 72% one is selected as it is the only one between Min (50%) and Max (75%), even though the 48% one is closer to the Goal (60%).

TABLE A
Example Sample Selection Chart

| | | Total | Live | CAR+ | Viabil- | Positiv- | Se- |
|---|---|---|---|---|---|---|---|
| CAR | Dilution | cell # | cell # | # | ity % | ity % | lected |
| CAR_1 | 1× | 57172 | 27850 | 25266 | 48.7 | 90.7 | |
| | 10× | 30775 | 15694 | 7805 | 51 | 49.7 | Yes |
| | 20× | 25195 | 12095 | 3398 | 48 | 28.1 | |
| | 40× | 20965 | 10736 | 1683 | 51.2 | 15.7 | |
| CAR_2 | 1× | 64945 | 36534 | 33905 | 56.3 | 92.8 | |
| | 10× | 46504 | 24319 | 20103 | 52.3 | 82.7 | |
| | 20× | 20369 | 10036 | 6036 | 49.3 | 60.1 | Yes |
| | 40× | 32520 | 16894 | 8839 | 51.9 | 52.3 | |

In accordance with one embodiment of the present disclosure, therefore, identification of selected samples entails comparing the expression levels from each of the M amounts to predetermined expression level cutoff values. In some embodiments, the predetermined expression level cutoff values comprise a maximum cutoff, a minimum cutoff and a goal cutoff.

In some embodiments, the method entails, for each vector:
(a) if the protein positivity levels of all M amounts are greater than the maximum cutoff, then the sample with the lowest protein positivity level is identified as the selected sample;
(b) if the protein positivity levels of all M amounts are lower than the minimum cutoff, then the sample with the highest protein positivity level is identified as the selected sample;
(c) if at least two of the protein positivity levels of all M amounts are between the maximum cutoff and the minimum cutoff, then the sample with a protein positivity level closest to the goal cutoff is identified as the selected sample; or
(d) if only one of the protein positivity levels of all M amounts is between the maximum cutoff and the minimum cutoff, then the sample having the protein positivity level between the maximum cutoff and the minimum cutoff is identified as the selected sample.

In some embodiments, all samples not identified as selected samples are not cultured together with the selected samples.

### Normalization

At various steps of the process, the cell numbers may be normalized to improve process consistency and efficiency. For instance, prior to cell transduction, the number of cells in each sample can be normalized to a predetermined number or concentration. In some embodiment, the predetermined concentration is 1×10⁵ cells/mL, 5×10⁵ cells/mL, 1×10⁶ cells/mL, 5×10⁶ cells/mL or 1×10⁷ cells/mL, without limitation.

The vector samples may also be normalized, in some embodiments. For instance, all vectors may be normalized to have the same concentration at the initial dilution (1×).

Once the vectors with the optional dilution level (e.g., 10×) are selected, in some embodiments, all of the selected samples are normalized. In one embodiment, the normalization is based on CAR positivity so that, after normalization, each sample has the same number of CAR-positive T cells. For instance, some of the T cells are removed from samples that have higher CAR positivity.

The selected, preferably normalized, CAR-T or TCR-T cell samples can be used for subsequent processing and analysis. For instance, a batch of CAR constructs can include different antibodies to the same antigen. Through such high-throughput screening, the CAR construct with the best antibody can be selected for clinical development. In another example, a batch of CAR constructs with different hinge domains can be screened and the identified optimal hinge domain can be used for further CAR development.

In some embodiments, the CAR or TCR includes an antibody or antigen binding molecule. The antigen binding molecule, in some embodiments, has binding specificity to an antigenic moiety. In some aspects, the antigenic moiety is associated with a cancer or a cancer cell. Such antigenic moieties may include, but are not limited to, 707-AP (707 alanine proline), AFP (alpha (a)-fetoprotein), ART-4 (adenocarcinoma antigen recognized by T4 cells), BAGE (B antigen; b-catenin/m, b-catenin/mutated), BCMA (B cell maturation antigen), Bcr-abl (breakpoint cluster region-Abelson), CAIX (carbonic anhydrase IX), CD19 (cluster of differentiation 19), CD20 (cluster of differentiation 20), CD22 (cluster of differentiation 22), CD30 (cluster of differentiation 30), CD33 (cluster of differentiation 33), CD44v7/8 (cluster of differentiation 44, exons 7/8), CAMEL (CTL-recognized antigen on melanoma), CAP-1 (carcinoembryonic antigen peptide-1), CASP-8 (caspase-8), CDC27m (cell-division cycle 27 mutated), CDK4/m (cycline-dependent kinase 4 mutated), CEA (carcinoembryonic antigen), CT (cancer/testis (antigen)), Cyp-B (cyclophilin B), DAM (differentiation antigen melanoma), EGFR (epidermal growth factor receptor), EGFRvIII (epidermal growth factor receptor, variant III), EGP-2 (epithelial glycoprotein 2), EGP-40 (epithelial glycoprotein 40), Erbb2, 3, 4 (erythroblastic leukemia viral oncogene homolog-2,-3, 4), ELF2M (elongation factor 2 mutated), ETV6-AML1 (Ets variant gene 6/acute myeloid leukemia 1 gene ETS), FBP (folate binding protein), fAchR (Fetal acetylcholine receptor), G250 (glycoprotein 250), GAGE (G antigen), GD2 (disialoganglioside 2), GD3 (disialoganglioside 3), GnT-V (N-acetylglucosaminyltransferase V), Gp100 (glycoprotein 100 kD), HAGE (helicose antigen), HER-2/neu (human epidermal receptor-2/neurological; also known as EGFR2), HLA-A (human leukocyte antigen-A) HPV (human papilloma virus), HSP70-2M (heat shock protein 70-2 mutated), HST-2 (human signet ring tumor-2), hTERT or hTRT (human telomerase reverse transcriptase), iCE (intestinal carboxyl esterase), IL-13R-a2 (Interleukin-13 receptor subunit alpha-2), KIAA0205, KDR (kinase insert domain receptor), κ-light chain, LAGE (L antigen), LDLR/FUT (low density lipid receptor/GDP-L-fucose: b-D-galactosidase 2-a-Lfucosyltransferase), LeY (Lewis-Y antibody), LICAM (L1 cell adhesion molecule), MAGE (melanoma antigen), MAGE-A1 (Melanoma-associated antigen 1), mesothelin, Murine CMV infected cells, MART-1/Melan-A (melanoma antigen recognized by T cells-1/Melanoma antigen A), MC1R (melanocortin 1 receptor), Myosin/m (myosin mutated), MUC1 (mucin 1), MUM-1,-2,-3 (melanoma ubiquitous mutated 1, 2, 3), NA88-A (NA cDNA clone of patient M88), NKG2D (Natural killer group 2, member D) ligands, NY-BR-1 (New York breast differentiation antigen 1), NY-ESO-1 (New York esophageal squamous cell carcinoma-1), oncofetal antigen (h5T4), P15 (protein 15), p190 minor bcr-abl (protein of 190KD bcr-abl), Pml/RARa (promyelocytic leukaemia/retinoic acid receptor a), PRAME (preferentially expressed antigen of melanoma), PSA (prostate-specific antigen), PSCA (Prostate stem cell antigen), PSMA (prostate-specific membrane antigen), RAGE (renal antigen), RU1 or RU2 (renal ubiquitous 1 or 2), SAGE (sarcoma antigen), SART-1 or SART-3 (squamous antigen rejecting tumor 1 or 3), SSX1,-2,-3, 4 (synovial sarcoma X1,-2,-3,-4), TAA (tumor-associated antigen), TAG-72 (Tumor-associated glycoprotein 72), TEL/AML1 (translocation Ets-family leukemia/acute myeloid leukemia 1), TPI/m (triosephosphate isomerase mutated), TRP-1 (tyrosinase related protein 1, or gp75), TRP-2 (tyrosinase related protein 2), TRP-2/INT2 (TRP-2/intron 2), VEGF-R2 (vascular endothelial growth factor receptor 2), or WT1 (Wilms' tumor gene).

Additional examples of cancer cell-associated antigens include 2B4 (CD244), 4-1BB, 5T4, A33 antigen, adenocarcinoma antigen, adrenoceptor beta 3 (ADRB3), A kinase anchor protein 4 (AKAP-4), alpha-fetoprotein (AFP), anaplastic lymphoma kinase (ALK), Androgen receptor, B7H3 (CD276), β2-integrins, BAFF, B-lymphoma cell, B cell maturation antigen (BCMA), bcr-abl (oncogene fusion protein consisting of breakpoint cluster region (BCR) and Abelson murine leukemia viral oncogene homolog 1 (Abl), BhCG, bone marrow stromal cell antigen 2 (BST2), CCCTC-Binding Factor (Zinc Finger Protein)-Like (BORIS or Brother of the Regulator of Imprinted Sites), BST2, C242 antigen, 9-0-acetyl-CA19-9 marker, CA-125, CAEX, calreticulin, carbonic anhydrase 9 (CAIX), C-MET, CCR4, CCR5, CCR8, CD2, CD3, CD4, CD5, CD8, CD7, CD10, CD16, CD19, CD20, CD22, CD23 (IgE receptor), CD24, CD25, CD27, CD28, CD30 (TNFRSF8), CD33, CD34, CD38, CD40, CD40L, CD41, CD44, CD44V6, CD49f, CD51, CD52, CD56, CD63, CD70, CD72, CD74, CD79a, CD79b, CD80, CD84, CD96, CD97, CD100, CD123, CD125, CD133, CD137, CD138, CD150, CD152 (CTLA-4), CD160, CD171, CD179a, CD200, CD221, CD229, CD244, CD272 (BTLA), CD274 (PDL-1, B7H1), CD279 (PD-1), CD352, CD358, CD300 molecule-like family member f (CD300LF), Carcinoembryonic antigen (CEA), claudin 6 (CLDN6), C-type lectin-like molecule-1 (CLL-1 or CLECL1), C-type lectin domain family 12 member A (CLEC12A), a cytomegalovirus (CMV) infected cell antigen, CNT0888, CRTAM (CD355), CS-1 (also referred to as CD2 subset 1, CRACC, CD319, and 19A24), CTLA-4, Cyclin B 1, chromosome X open reading frame 61 (CXORF61), Cytochrome P450 1B 1 (CYP1B1), DNAM-1 (CD226), desmoglein 4, DR3, DR5, E-cadherin neoepitope, epidermal growth factor receptor (EGFR), EGFR, epidermal growth factor receptor variant III (EGFRvIII), epithelial glycoprotein-2 (EGP-2), epithelial glycoprotein-40 (EGP-40), EGF-like module-containing mucin-like hormone receptor-like 2 (EMR2), elongation factor 2 mutated (ELF2M), endosialin, Epithelial cell adhesion molecule (EPCAM), ephrin type-A receptor 2 (EphA2), Ephrin B2, receptor tyrosine-protein kinases erb-B2,3,4 (erb-B2,3,4), ERBB, ERBB2 (Her2/neu), ERG (transmembrane protease, serine 2 (TMPRSS2) ETS fusion gene), ETA, ETS translocation-variant gene 6, located on chromosome 12p (ETV6-AML), Fc fragment of IgA receptor (FCAR or CD89), fibroblast activation protein alpha (FAP), FBP, Fc receptor-like 5 (FCRL5), fetal acetylcholine receptor (AChR), fibronectin extra domain-B, Fms-Like Tyrosine Kinase 3 (FLT3), folate-binding protein (FBP), folate receptor 1, folate receptor α, Folate receptor β, Fos-related antigen 1, Fucosyl, Fucosyl GM1; GM2, ganglioside G2 (GD2), ganglioside GD3 (aNeu5Ac (2-8) aNeu5Ac (2-3) bDGalp (1-4) bDGlcp (1-1) Cer),o-acetyl-GD2 ganglioside (OAcGD2), GITR (TNFRSF 18), GM1, ganglioside GM3 (aNeu5Ac (2-3) bDGalp (1-4) bDGlcp (1-1) Cer), GP 100, hexasaccharide portion of globoH glycoceramide (GloboH), glycoprotein 75, Glypican-3 (GPC3), glycoprotein 100 (gplOO), GPNMB, G protein-coupled receptor 20 (GPR20), G protein-coupled receptor class C group 5, member D (GPRC5D), Hepatitis A virus cellular receptor 1 (HAVCR1), human Epidermal Growth Factor Receptor 2 (HER-2), HER2/neu, HER3, HER4, HGF, high molecular weight-melanoma-associated antigen (HMWMAA), human papilloma virus E6 (HPV E6), human papilloma virus E7 (HPV E7), heat shock protein 70-2 mutated (mut hsp70-2), human scatter factor receptor kinase, human Telomerase reverse transcriptase (hTERT), HVEM, ICOS, insulin-like growth factor receptor 1 (IGF-1 receptor), IGF-I, IgG1, immunoglobulin lambda-like polypeptide 1 (IGLL1), IL-6, Interleukin 11 receptor alpha (IL-11Ra), IL-13, Interleukin-13 receptor subunit alpha-2 (IL-13Ra2 or CD213A2), insulin-like growth factor I receptor (IGF1-R), integrin α5β1, integrin ανβ3, intestinal carboxyl esterase, k-light chain, KCS1, kinase insert domain receptor (KDR), KIR, KIR2DL1, KIR2DL2, KIR2DL3, KIR3DL2, KIR-L, KG2D ligands, KIT (CD117), KLRGI, LAGE-la, LAG3, lymphocyte-specific protein tyrosine kinase (LCK), Leukocyte immunoglobulin-like receptor subfamily A member 2 (LILRA2), legumain, Leukocyte-associated immunoglobulin-like receptor 1 (LAIR1), Lewis (Y) antigen, LeY, LG, LI cell adhesion molecule (LI-CAM), LIGHT, LMP2, lymphocyte antigen 6 complex, LTBR, locus K 9 (LY6K), Ly-6, lymphocyte antigen 75 (LY75), melanoma cancer testis antigen-1 (MAD-CT-1); melanoma cancer testis antigen-2 (MAD-CT-2), MAGE, Melanoma-associated antigen 1 (MAGE-A1), MAGE-A3 melanoma antigen recognized by T cells 1 (MelanA or MARTI), MelanA/MARTI, Mesothelin, MAGE A3, melanoma inhibitor of apoptosis (ML-IAP), melanoma-specific chondroitin-sulfate proteoglycan (MCSCP), MORAb-009, MS4A1, Mucin 1 (MUCI), MUC2, MUC3, MUC4, MUC5AC, MUC5b, MUC7, MUC16, mucin CanAg, Mullerian inhibitory substance (MIS) receptor type II, v-myc avian myelocytomatosis viral oncogene neuroblastoma derived homolog (MYCN), N-glycolylneuraminic acid, N-Acetyl glucosaminyl-transferase V (NA17), neural cell adhesion molecule (NCAM), NKG2A, NKG2C, NKG2D, NKG2E ligands, NKR-P IA,NPC-1C, NTB-A, mammary gland differentiation antigen (NY-BR-1), NY-ESO-1, oncofetal antigen (h5T4), Olfactory receptor 51E2 (OR51E2), OX40, plasma cell antigen, poly SA, proacrosin binding protein sp32 (OY-TES 1), p53, p53 mutant, pannexin 3 (PANX3), prostatic acid phosphatase (PAP), paired box protein Pax-3 (PAX3), Paired box protein Pax-5 (PAX5), prostate carcinoma tumor antigen-1 (PCTA-1 or Galectin 8), PD-1H, Platelet-derived growth factor receptor alpha (PDGFR-alpha), PDGFR-beta, PDL192, PEN-5, phosphatidylserine, placenta-specific 1 (PLAC1), Polysialic acid, Prostase, prostatic carcinoma cells, prostein, Protease Serine 21 (Testisin or PRSS21), Proteinase3 (PR1), prostate stem cell antigen (PSCA), prostate-specific membrane antigen (PSMA), Proteasome (Prosome, Macropain) Subunit, Beta Type, Receptor for Advanced Glycation Endproducts (RAGE-1), RANKL, Ras mutant, Ras Homolog Family Member C (RhoC), RON, Receptor tyrosine kinase-like orphan receptor 1 (ROR1), renal ubiquitous 1 (RU1), renal ubiquitous 2 (RU2), sarcoma translocation breakpoints, Squamous Cell Carcinoma Antigen Recognized By T Cells 3 (SART3), SAS, SDC1, SLAMF7, sialyl Lewis adhesion molecule (sLe), Siglec-3, Siglec-7, Siglec-9, sonic hedgehog (SHH), sperm protein 17 (SPA17), Stage-specific embryonic antigen-4 (SSEA-4), STEAP, sTn antigen, synovial sarcoma, X breakpoint 2 (SSX2), Survivin, Tumor-associated glycoprotein 72 (TAG72), TCR5y, TCRa, TCRB, TCR Gamma Alternate Reading Frame Protein (TARP), telomerase, TIGIT TNF-α precursor, tumor endothelial marker 1 (TEM1/CD248), tumor endothelial marker 7-related (TEM7R), tenascin C, TGF beta 2, TGF-β, transglutaminase 5 (TGS5), angiopoietin-binding cell surface receptor 2 (Tie 2), TIM1, TIM2, TIM3, Tn Ag, TRAIL-R1, TRAIL-R2, Tyrosinase-related protein 2 (TRP-2), thyroid stimulating hormone receptor (TSHR), tumor antigen CTAA16.88, Tyrosinase, ROR1, TAG-72, uroplakin 2 (UPK2), VEGF-A, VEGFR-1, vascular endothelial growth factor receptor 2 (VEGFR2), and vimentin, Wilms tumor protein (WT1), or X Antigen Family, Member 1A (XAGE1).

In other embodiments, the antigenic moiety is associated with virally infected cells (i.e., a viral antigenic moiety). Such antigenic moieties may include, but are not limited to, an Epstein-Barr virus (EBV) antigen (e.g., EBNA-1, EBNA-2, EBNA-3, LMP-1, LMP-2), a hepatitis A virus antigen (e.g., VP1, VP2, VP3), a hepatitis B virus antigen (e.g., HBsAg, HBcAg, HBeAg), a hepatitis C viral antigen (e.g., envelope glycoproteins E1 and E2), a herpes simplex virus type 1, type 2, or type 8 (HSV1, HSV2, or HSV8) viral antigen (e.g., glycoproteins gB, gC, gC, gE, gG, gH, gI, gJ, gK, gL. gM, UL20, UL32, US43, UL45, UL49A), a cytomegalovirus (CMV) viral antigen (e.g., glycoproteins gB, gC, gC, gE, gG, gH, gI, gJ, gK, gL. gM or other envelope proteins), a human immunodeficiency virus (HIV) viral antigen (glycoproteins gp120, gp41, or p24), an influenza viral antigen (e.g., hemagglutinin (HA) or neuraminidase (NA)), a measles or mumps viral antigen, a human papillomavirus (HPV) viral antigen (e.g., L1, L2), a parainfluenza virus viral antigen, a rubella virus viral antigen, a respiratory syncytial virus (RSV) viral antigen, or a varicella-zostser virus viral antigen. In such embodiments, the cell surface receptor may be any TCR, or any CAR which recognizes any of the aforementioned viral antigens on a target virally infected cell.

In other embodiments, the antigenic moiety is associated with cells having an immune or inflammatory dysfunction. Such antigenic moieties may include, but are not limited to, myelin basic protein (MBP) myelin proteolipid protein (PLP), myelin oligodendrocyte glycoprotein (MOG), carcinoembryonic antigen (CEA), pro-insulin, glutamine decarboxylase (GAD65, GAD67), heat shock proteins (HSPs), or any other tissue specific antigen that is involved in or associated with a pathogenic autoimmune process.

In some embodiments, the TCR has specificity to an antigenic moiety on cancer cells. Non-limiting examples of TCR include an anti-707-AP TCR, anti-AFP TCR, anti-ART-4 TCR, anti-BAGE TCR, anti-Bcr-abl TCR, anti-CAMEL TCR, anti-CAP-1 TCR, anti-CASP-8 TCR, anti-CDC27m TCR, anti-CDK4/m TCR, anti-CEA TCR, anti-CT TCR, anti-Cyp-B TCR, anti-DAM TCR, anti-TCR, anti-EGFRvIII TCR, anti-ELF2M TCR, anti-ETV6-AML1 TCR, anti-G250 TCR, GAGE TCR, anti-GnT-V TCR, anti-Gp100 TCR, anti-HAGE TCR, anti-HER-2/neu TCR, anti-HLA-A TCR, anti-HPV TCR, anti-HSP70-2M TCR, anti-HST-2 TCR, anti-hTERT TCR or anti-hTRT TCR, anti-iCE TCR, anti-KIAA0205, anti-LAGE (L antigen), anti-LDLR/FUT TCR, anti-MAGE TCR, anti-MART-1/Melan-A TCR, anti-MC1R TCR, anti-Myosin/m TCR, anti-MUC1 TCR, anti-MUM-1,-2,-3 TCR, anti-NA88-A TCR, anti-NY-ESO-1 TCR, anti-P15 TCR, anti-p190 minor bcr-abl TCR, anti-Pml/RARa TCR, anti-PRAME TCR, anti-PSA TCR, anti-PSMA TCR, anti-RAGE TCR, anti-RU1 TCR or anti-RU2 TCR, anti-SAGE TCR, anti-SART-1 TCR or anti-SART-3 TCR, anti-SSX1,-2,-3, 4 TCR, anti-TEL/AML1 TCR, anti-TPI/m TCR, anti-TRP-1 TCR, anti-TRP-2 TCR, anti-TRP-2/INT2 TCR, or anti-WT1 TCR.

A CAR of the present disclosure can include, in addition to the antigen-binding molecule, a hinge, a transmembrane domain, and/or an intracellular domain. In some embodiments, the intracellular domain can include a costimulatory domain and an activation domain.

A hinge may be an extracellular domain of an antigen binding system positioned between the binding motif and the transmembrane domain. A hinge may also be referred to as an extracellular domain or as a “spacer.” A hinge may contribute to receptor expression, activity, and/or stability. A hinge may also provide flexibility to access the targeted antigen. In some embodiments, a hinge domain is positioned between a binding motif and a transmembrane domain.

In some embodiments, the hinge is, is from, or is derived from (e.g., comprises all or a fragment of) an immunoglobulin-like hinge domain. In some embodiments, a hinge domain is from or derived from an immunoglobulin. In some embodiments, a hinge domain is selected from the hinge of IgG1, IgG2, IgG3, IgG4, IgA, IgD, IgE, or IgM, or a fragment thereof.

In some embodiments, the hinge is, is from, or is derived from (e.g., comprises all or a fragment of) CD2, CD3 delta, CD3 epsilon, CD3 gamma, CD4, CD7, CD8.alpha., CD8.beta., CD11a (ITGAL), CD11b (ITGAM), CD11c (ITGAX), CD11d (ITGAD), CD18 (ITGB2), CD19 (B4), CD27 (TNFRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD100 (SEMA4D), CD103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3-zeta), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulator (ICOS), LFA-1 (CD11a/CD18), NKG2C, DAP-10, ICAM-1, NKp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, or Toll ligand receptor, or which is a fragment or combination thereof.

In some embodiments, the hinge is, is from, or is derived from (e.g., comprises all or a fragment of) a hinge of CD8 alpha. In some embodiments, the hinge is, is from, or is derived from a hinge of CD28. In some embodiments, the hinge is, is from, or is derived from a fragment of a hinge of CD8 alpha or a fragment of a hinge of CD28, wherein the fragment is anything less than the whole. In some embodiments, a fragment of a CD8 alpha hinge or a fragment of a CD28 hinge comprises an amino acid sequence that excludes at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids at the N-terminus or C-Terminus, or both, of a CD8 alpha hinge, or of a CD28 hinge.

A “transmembrane domain” refers to a domain having an attribute of being present in the membrane when present in a molecule at a cell surface or cell membrane (e.g., spanning a portion or all of a cellular membrane). It is not required that every amino acid in a transmembrane domain be present in the membrane. For example, in some embodiments, a transmembrane domain is characterized in that a designated stretch or portion of a protein is substantially located in the membrane. Amino acid or nucleic acid sequences may be analyzed using a variety of algorithms to predict protein subcellular localization (e.g., transmembrane localization). The programs psort (PSORT.org) and Prosite (prosite.expasy.org) are exemplary of such programs.

A transmembrane domain may be derived either from any membrane-bound or transmembrane protein, such as an alpha, beta or zeta chain of a T-cell receptor, CD28, CD3 epsilon, CD3 delta, CD3 gamma, CD45, CD4, CD5, CD7, CD8, CD8 alpha, CD8beta, CD9, CD11a, CD11b, CD11c, CD11d, CD16, CD22, CD27, CD33, CD37, CD64, CD80, CD86, CD134, CD137, TNFSFR25, CD154, 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD276 (B7-H3), CD29, CD30, CD40, CD49a, CD49D, CD49f, CD69, CD84, CD96 (Tactile), CD5, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, a ligand that binds with CD83, LIGHT, LIGHT, LTBR, Ly9 (CD229), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A; Ly108), SLAMF7, SLP-76, TNF receptor proteins, TNFR2, TNFSF14, a Toll ligand receptor, TRANCE/RANKL, VLA1, or VLA-6, or a fragment, truncation, or a combination thereof.

The intracellular domain (or cytoplasmic domain) comprises one or more signaling domains that, upon binding of target antigen to the binding motif, cause and/or mediate an intracellular signal, e.g., that activates one or more immune cell effector functions (e.g., native immune cell effector functions). In some embodiments, signaling domains of an intracellular domain mediate activation at least one of the normal effector functions of the immune cell. Effector function of a T cell, for example, may be cytolytic activity or helper activity comprising the secretion of cytokines. In some embodiments, signaling domains of an intracellular domain mediate T cell activation, proliferation, survival, and/or other T cell function. An intracellular domain may comprise a signaling domain that is an activating domain. An intracellular domain may comprise a signaling domain that is a costimulatory signaling domain.

Intracellular signaling domains that may transduce a signal upon binding of an antigen to an immune cell are known. For example, cytoplasmic sequences of a T cell receptor (TCR) are known to initiate signal transduction following TCR binding to an antigen (see, e.g., Brownlie et al., Nature Rev. Immunol. 13:257-269 (2013)).

In certain embodiments, suitable signaling domains include, without limitation, those of 4-1BB/CD137, activating NK cell receptors, an Immunoglobulin protein, B7-H3, BAFFR, BLAME (SLAMF8), BTLA, CD100 (SEMA4D), CD103, CD160 (BY55), CD18, CD19, CD19a, CD2, CD247, CD27, CD276 (B7-H3), CD28, CD29, CD3 delta, CD3 epsilon, CD3 gamma, CD30, CD4, CD40, CD49a, CD49D, CD49f, CD69, CD7, CD84, CD8alpha, CD8beta, CD96 (Tactile), CD11a, CD11b, CD11c, CD11d, CD5, CEACAM1, CRT AM, cytokine receptor, DAP-10, DNAM1 (CD226), Fc gamma receptor, GADS, GITR, HVEM (LIGHTR), IA4, ICAM-1, ICAM-1, Ig alpha (CD79a), IL-2R beta, IL-2R gamma, IL-7R alpha, inducible T cell costimulator (ICOS), integrins, ITGA4, ITGA4, ITGA6, ITGAD, ITGAE, ITGAL, ITGAM, ITGAX, ITGB2, ITGB7, ITGB1, KIRDS2, LAT, LFA-1, LFA-1, ligand that binds with CD83, LIGHT, LIGHT, LTBR, Ly9 (CD229), Ly108), lymphocyte function-associated antigen-1 (LFA-1; CD1-1a/CD18), MHC class 1 molecule, NKG2C, NKG2D, NKp30, NKp44, NKp46, NKp80 (KLRF1), OX-40, PAG/Cbp, programmed death-1 (PD-1), PSGL1, SELPLG (CD162), Signaling Lymphocytic Activation Molecules (SLAM proteins), SLAM (SLAMF1; CD150; IPO-3), SLAMF4 (CD244; 2B4), SLAMF6 (NTB-A, SLAMF7, SLP-76, TNF receptor proteins, TNFR2, TNFSF14, a Toll ligand receptor, TRANCE/RANKL, VLA1, or VLA-6, or a fragment, truncation, or a combination thereof.

A CAR can also include a costimulatory signaling domain, e.g., to increase signaling potency. See U.S. Pat. Nos. 7,741,465, and 6,319,494, as well as Krause et al. and Finney et al. (supra), Song et al., Blood 119:696-706 (2012); Kalos et al., Sci Transl. Med. 3:95 (2011); Porter et al., N. Engl. J. Med. 365:725-33 (2011), and Gross et al., Annu. Rev. Pharmacol. Toxicol. 56:59-83 (2016). Signals generated through a TCR alone may be insufficient for full activation of a T cell and a secondary or co-stimulatory signal may increase activation. Thus, in some embodiments, a signaling domain further comprises one or more additional signaling domains (e.g., costimulatory signaling domains) that activate one or more immune cell effector functions (e.g., a native immune cell effector function described herein). In some embodiments, a portion of such costimulatory signaling domains may be used, as long as the portion transduces the effector function signal. In some embodiments, a cytoplasmic domain described herein comprises one or more cytoplasmic sequences of a T cell co-receptor (or fragment thereof). Non-limiting examples of such T cell co-receptors comprise CD27, CD28, 4-1BB (CD137), OX40, CD30, CD40, PD-1, ICOS, lymphocyte function-associated antigen-1 (LFA-1), MYD88, CD2, CD7, LIGHT, NKG2C, B7-H3, and a ligand that binds with CD83. An exemplary costimulatory protein has the amino acid sequence of a costimulatory protein found naturally on T cells, the complete native amino acid sequence of which costimulatory protein is described in NCBI Reference Sequence: NP 0.1. In certain instances, a CAR includes a 4-1BB costimulatory domain. In certain instances, a CAR includes a CD28 costimulatory domain. In certain instances, a CAR includes a DAP-10 costimulatory domain.

In some embodiments, the costimulatory signaling domain is a signaling domain of CD28. As shown in the experimental examples, CAR molecules with a CD28 costimulatory signaling domain can particularly benefit from the newly developed, expedited manufacturing process.

In some embodiments, the CAR further includes an ITAM. Examples of ITAM containing primary cytoplasmic signaling sequences that are of particular use in the disclosure include those derived from TCRzeta, FcRgamma, FcRbeta, CD3gamma, CD3delta, CD3epsilon, CD5, CD22, CD79a, CD79b and CD66d. In some embodiments, the ITAM includes CD3 zeta.

In some embodiments, a pharmaceutical composition is provided that includes a population of engineered lymphocytes produced by the methods described herein. In certain embodiments, the pharmaceutical composition may also include a pharmaceutically acceptable carrier. A pharmaceutically acceptable carrier may be a pharmaceutically acceptable material, composition, or vehicle that is involved in carrying or transporting cells of interest from one tissue, organ, or portion of the body to another tissue, organ, or portion of the body. For example, the carrier may be a liquid or solid filler, diluent, excipient, solvent, or encapsulating material, or some combination thereof. Each component of the carrier must be “pharmaceutically acceptable” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits.

### Treatments and Uses, and Optional Storage

The T cells selected or prepared by the instant methods can be used for treating various diseases and conditions.

In some embodiments, if the T cells are not immediately used, they can cryopreserved so that they can be used at a later date. Such a method may include a step of washing and concentrating the population of engineered lymphocytes with a diluent solution. In some aspects the diluent solution is normal saline, 0.9% saline, PlasmaLyte A (PL), 5% dextrose/0.45% NaCl saline solution (D5), human serum albumin (HSA), or a combination thereof. In some aspects, HSA may be added to the washed and concentrated cells for improved cell viability and cell recovery after thawing. In another aspect, the washing solution is normal saline and washed and concentrated cells are supplemented with HSA (5%). The method may also include a step of generating a cryopreservation mixture, wherein the cryopreservation mixture includes the diluted population of cells in the diluent solution and a suitable cryopreservative solution. In some aspects, the cryopreservative solution may be any suitable cryopreservative solution including, but not limited to, CryoStor10 (BioLife Solution), mixed with the diluent solution of engineered lymphocytes at a ratio of 1:1 or 2:1.

In certain embodiments, HSA may be added to provide a final concentration of about 1.0-10% HSA in the cryopreserved mixture. In certain embodiments, HSA may be added to provide a final concentration of about 1.0%, about 2.0%, about 3.0%, about 4.0%, about 5.0%, about 6.0%, about 7.0%, about 8.0%, about 9.0%, or about 10.0% HSA in the cryopreserved mixture. In certain embodiments, HSA may be added to provide a final concentration of about 1-3% HSA, about 1-4% HSA, about 1-5% HSA, about 1-7% HSA, about 2-4% HSA, about 2-5% HSA, about 2-6% HSA, or about 2-7% HSA in the cryopreserved mixture. In certain embodiments, HSA may be added to provide a final concentration of about 2.5% HSA in the cryopreserved mixture. For example, in certain embodiments, cryopreservation of a population of engineered T cells may comprise washing cells with 0.9% normal saline, adding HSA at a final concentration of 5% to the washed cells, and diluting the cells 1:1 with CryoStor™ CS10 (for a final concentration of 2.5% HSA in the final cryopreservation mixture). In some embodiments, the method also includes a step of freezing the cryopreservation mixture. In one aspect, the cryopreservation mixture is frozen in a controlled rate freezer using a defined freeze cycle at a cell concentration of between about 1e6 to about 1.5e7 cells per mL of cryopreservation mixture. The method may also include a step of storing the cryopreservation mixture in vapor phase liquid nitrogen.

Methods and uses are also provided, for treating a disease or pathological condition in a subject having the disease or pathological condition. In some embodiments, the method entails administering a therapeutically effective amount or therapeutically effective dose of the engineered T cells to the subject. Pathogenic conditions that may be treated with engineered T cells that are produced by the methods described herein include, but are not limited to, cancer, viral infection, acute or chronic inflammation, autoimmune disease or any other immune-dysfunction.

As referred to herein, a “cancer” may be any cancer that is associated with a surface antigen or cancer marker, including, but not limited to, acute lymphoblastic leukemia (ALL), acute myeloid leukemia (AML), adenoid cystic carcinoma, adrenocortical, carcinoma, AIDS-related cancers, anal cancer, appendix cancer, astrocytomas, atypical teratoid/rhabdoid tumor, central nervous system, B-cell leukemia, lymphoma or other B cell malignancies, basal cell carcinoma, bile duct cancer, bladder cancer, bone cancer, osteosarcoma and malignant fibrous histiocytoma, brain stem glioma, brain tumors, breast cancer, bronchial tumors, burkitt lymphoma, carcinoid tumors, central nervous system cancers, cervical cancer, chordoma, chronic lymphocytic leukemia (CLL), chronic myelogenous leukemia (CML), chronic myeloproliferative disorders, colon cancer, colorectal cancer, craniopharyngioma, cutaneous t-cell lymphoma, embryonal tumors, central nervous system, endometrial cancer, ependymoblastoma, ependymoma, esophageal cancer, esthesioneuroblastoma, ewing sarcoma family of tumors extracranial germ cell tumor, extragonadal germ cell tumor extrahepatic bile duct cancer, eye cancer fibrous histiocytoma of bone, malignant, and osteosarcoma, gallbladder cancer, gastric (stomach) cancer, gastrointestinal carcinoid tumor, gastrointestinal stromal tumors (GIST), soft tissue sarcoma, germ cell tumor, gestational trophoblastic tumor, glioma, hairy cell leukemia, head and neck cancer, heart cancer, hepatocellular (liver) cancer, histiocytosis, hodgkin lymphoma, hypopharyngeal cancer, intraocular melanoma, islet cell tumors (endocrine pancreas), kaposi sarcoma, kidney cancer, langerhans cell histiocytosis, laryngeal cancer, leukemia, lip and oral cavity cancer, liver cancer (primary), lobular carcinoma in situ (LCIS), lung cancer, lymphoma, macroglobulinemia, male breast cancer, malignant fibrous histiocytoma of bone and osteosarcoma, medulloblastoma, medulloepithelioma, melanoma, merkel cell carcinoma, mesothelioma, metastatic squamous neck cancer with occult primary midline tract carcinoma involving NUT gene, mouth cancer, multiple endocrine neoplasia syndromes, multiple myeloma/plasma cell neoplasm, mycosis fungoides, myelodysplastic syndromes, myelodysplastic/myeloproliferative neoplasms, myelogenous leukemia, chronic (CML), Myeloid leukemia, acute (AML), myeloma, multiple, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma of bone, ovarian cancer, pancreatic cancer, papillomatosis, paraganglioma, paranasal sinus and nasal cavity cancer, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm/multiple myeloma, pleuropulmonary blastoma, pregnancy and breast cancer, primary central nervous system (CNS) lymphoma, prostate cancer, rectal cancer, renal cell (kidney) cancer, renal pelvis and ureter, transitional cell cancer, retinoblastoma, rhabdomyosarcoma, salivary gland cancer, sarcoma, sézary syndrome, small cell lung cancer, small intestine cancer, soft tissue sarcoma, squamous cell carcinoma, squamous neck cancer, stomach (gastric) cancer, supratentorial primitive neuroectodermal tumors, t-cell lymphoma, cutaneous, testicular cancer, throat cancer, thymoma and thymic carcinoma, thyroid cancer, transitional cell cancer of the renal pelvis and ureter, trophoblastic tumor, ureter and renal pelvis cancer, urethral cancer, uterine cancer, uterine sarcoma, vaginal cancer, vulvar cancer, Waldenström macroglobulinemia, Wilms Tumor.

In some aspects, the cancer is a B cell malignancy. Examples of B cell malignancies include, but are not limited to, Non-Hodgkin's Lymphomas (NHL), Diffuse Large B Cell Lymphoma (DLBCL), Small lymphocytic lymphoma (SLL/CLL), Mantle cell lymphoma (MCL), Follicular lymphoma (FL), Marginal zone lymphoma (MZL), Extranodal (MALT lymphoma), Nodal (Monocytoid B-cell lymphoma), Splenic, Diffuse large cell lymphoma, B cell chronic lymphocytic leukemia/lymphoma, Burkitt's lymphoma and Lymphoblastic lymphoma.

As referred to herein, a “viral infection” may be an infection caused by any virus which causes a disease or pathological condition in the host. Examples of viral infections that may be treated with the engineered T cells that are produced by the methods described herein include, but are not limited to, a viral infection caused by an Epstein-Barr virus (EBV); a viral infection caused by a hepatitis A virus, a hepatitis B virus or a hepatitis C virus; a viral infection caused by a herpes simplex type 1 virus, a herpes simplex type 2 virus, or a herpes simplex type 8 virus, a viral infection caused by a cytomegalovirus (CMV), a viral infection caused by a human immunodeficiency virus (HIV), a viral infection caused by an influenza virus, a viral infection caused by a measles or mumps virus, a viral infection caused by a human papillomavirus (HPV), a viral infection caused by a parainfluenza virus, a viral infection caused by a rubella virus, a viral infection caused by a respiratory syncytial virus (RSV), or a viral infection caused by a varicella-zostser virus. In some aspects, a viral infection may lead to or result in the development of cancer in a subject with the viral infection (e.g., HPV infection may cause or be associated with the development of several cancers, including cervical, vulvar, vaginal, penile, anal, oropharyngeal cancers, and HIV infection may cause the development of Kaposi's sarcoma)

Examples of chronic inflammation diseases, autoimmune diseases or any other immune-dysfunctions that may be treated with the engineered T cells produced by the methods described herein include, but are not limited to, multiple sclerosis, lupus, and psoriasis.

The term “treat,” “treating” or “treatment” as used herein with regard to a condition or disease may refer to preventing a condition or disease, slowing the onset or rate of development of the condition or disease, reducing the risk of developing the condition or disease, preventing or delaying the development of symptoms associated with the condition or disease, reducing or ending symptoms associated with the condition or disease, generating a complete or partial regression of the condition or disease, or some combination thereof.

A “therapeutically effective amount” or a “therapeutically effective dose” is an amount of engineered lymphocytes that produce a desired therapeutic effect in a subject, such as preventing or treating a target condition or alleviating symptoms associated with the condition by killing target cells. The most effective results in terms of efficacy of treatment in a given subject will vary depending upon a variety of factors, including but not limited to the characteristics of the engineered lymphocytes (including longevity, activity, pharmacokinetics, pharmacodynamics, and bioavailability), the physiological condition of the subject (including age, sex, disease type and stage, general physical condition, responsiveness to a given dosage, and type of medication), the nature of any pharmaceutically acceptable carrier or carriers in any composition used, and the route of administration. A therapeutically effective dose of engineered lymphocytes also depends on the cell surface receptor that is expressed by the lymphocytes (e.g., the affinity and density of the cell surface receptors expressed on the cell), the type of target cell, the nature of the disease or pathological condition being treated, or a combination of both.

As shown in the examples, the engineered lymphocytes prepared by the instant process have greatly increased in vivo efficacy and thus a much lower dose is required, as compared to the conventional technology.

Therefore, in some aspects, a therapeutically effective dose of engineered lymphocytes is fewer than about 2 million engineered lymphocytes per kilogram of body weight (cells/kg). Therefore, in some aspects, a therapeutically effective dose of engineered lymphocytes is from about 10,000 to about 1,500,000 engineered lymphocytes/kg. In certain embodiments, the therapeutically effective dose is about 20,000 to about 1,200,000 million engineered lymphocytes/kg. In certain embodiments, the therapeutically effective dose is about 20,000 to about 1,000,000 million engineered lymphocytes/kg. In certain embodiments, the therapeutically effective dose is about 20,000 to about 500,000 million engineered lymphocytes/kg. In certain embodiments, the therapeutically effective dose is about 20,000 to about 400,000 million engineered lymphocytes/kg. In certain embodiments, the therapeutically effective dose is about 40,000 to about 400,000 million engineered lymphocytes/kg. In certain embodiments, the therapeutically effective dose is about 50,000 to about 200,000 million engineered lymphocytes/kg. In certain embodiments, the therapeutically effective dose is about 50,000 to about 100,000 million engineered lymphocytes/kg.

### Example 1: Automated High-Throughput Process for Manufacturing and Screening of CAR T Cells

This example tested a new, automated, high-throughput (HT) CAR-T cell manufacturing and screening process for the identification of tumor-specific antigen CAR-T cells.

### A. Transduction Efficiencies of Donor and Jurkat T Cells

First, this example compared the transduction efficiencies of primary T cells from donors and the Jurkat cell lines, using a number of diverse CAR lentiviral (LVV) constructs. Donor T cells from six different subjects were used. The transduction efficiencies (in transduction unit (TU)/mL) were plotted between the donor T cells and Jurkat cells (FIG. 1).

There was essentially no correlation of LVV titers between the donor cells and Jurkat cells. Generally, as shown in FIG. 1, the transduction efficiencies into Jurkat cells were low as compared to the donor cells. There was a high frequency of candidate dropouts due to poor CAR expression. Donor T cells, therefore, were used in subsequent testing.

### B. Donor T and Target Cell Noise

Second, a process was developed to identify suitable T cell donors prior to screening. To this end, multiple donor T cell candidates were tested against benchmark CARs and multiple target lines.

In addition, the cytotoxicity of the T cells with or without the transduced CAR was compared. As shown in FIG. 2, while the transduced CAR-T cells from most donors exhibited good cytotoxicity, some showed high background cytotoxicity even without the CAR transduced into the cells (noise). For subsequent CAR-T screening, donor T cells with the highest CAR-T cytotoxicity/noise ratios were used.

### C. CAR-T Screening

A high-throughput CAR-T screening process was developed. The general process is described as follows.

TABLE 1
Example CAR-T Screening Process

| Steps | Protocol | |
|---|---|---|
| Donor | Day −1: | |
| T cell | 1. | 96-well plates are coated with anti-CD3 antibody |
| activation | | overnight |
| | 2. | Tissue culture flask is coated with anti-CD3 antibody |
| | | (OKT3) overnight |
| | Day 0: | |
| | 1. | Donor T cells are thawed and normalized to 1 × 10⁶ |
| | | cells/mL in media |
| | 2. | Cells are transferred into an anti-CD3 antibody-coated |
| | | tissue culture flask |
| | 3. | Anti-CD28 antibody is added to the cells |
| | 4. | Cells are allowed to recover overnight into a 37° C. |
| | | 5% CO₂ incubator |
| LVV | Day 1: | |
| dilution | 1. | Virus is arrayed into 96-well plates |
| and | 2. | Virus is diluted 10×, 20×, and 40× in media |
| addition | 3. | Donor T cells are counted then normalized to 5 × |
| | | 10⁵ cells/mL in media with anti-CD28 antibody |
| | 4. | Normalized cells are plated into 96-well plates coated |
| | | with anti-CD3 antibody using an automated liquid |
| | | dispenser |
| | 5. | Viruses (diluted) are transferred to the plated cells |
| | | using liquid handling |
| | 6. | Cells are incubated with virus for 48 hours in a 37° |
| | | C. 5% CO₂ incubator |
| Transduc- | Day 3: | |
| tion | 1. | Randomly selected representative non-transduced cell |
| | | wells are counted |
| | 2. | Cell dilution calculated for final concentration of 1.25 × |
| | | 10⁵ cells/mL |
| | 3. | Media added to 96-well plates using automated liquid |
| | | dispenser |
| | 4. | Cells are transferred to multiple plates using an |
| | | automated liquid handling |
| | 5. | Cells are incubated with virus for 72 hours in a |
| | | 37° C. 5% CO₂ incubator |
| CAR | Day 6: | |
| positivity | 1. | Expanded cells in 96-well plates are consolidated into |
| determi- | | 96-well deep well plates using automated liquid handling |
| nation and | 2. | Aliquot of cells are analyzed for cell count and CAR |
| hit picking | | positivity using a high-throughput cell analyzer |
| | 3. | CAR T cells with optimal CAR positivity are hit picked |
| | | and normalized using automated liquid handling robot |
| Expansion | Day 6: | |
| of hits | 1. | Cell dilution calculated for final concentration of 5 × 10⁵ |
| | | cells/mL |
| | 2. | Media added to 96-well plates using an automated liquid |
| | | handling robot |
| | 3. | Cells are transferred to multiple plates using automated |
| | | liquid handling |
| | 4. | Cells are incubated with virus for 72 hours in a 37° C. |
| | | 5% CO₂ incubator |
| | Day 9: | |
| | 1. | Cells are used in a high throughput screening co-culture |
| | | assay |

As illustrated in the protocol, selected donor T cells were activated with anti-CD3 and anti-CD28 antibodies, and then subjected to viral transduction. Transduced T cells were then subjected to CAR positivity, cell count and cell viability measurements.

One objective of this screening process was to identify a suitable CAR-T sample for further testing and evaluation for each CAR construct in a corresponding donor T cell. For each CAR, which was provided in lentiviral (LVV) vectors, four different dilutions were used (1×, 10×, 20× and 40×). The CAR positivity and viability of each CAR-T was determined using a high-throughput cell analyzer.

Out of the four dilutions, the CAR-T sample from one of them was selected for further expansion and testing. An example set of selection criteria include three pre-determined cutoff values, (A) Minimum CAR positivity (“Min”), (B) Goal CAR positivity (“Goal”), and (C) Maximum CAR positivity (“Max”). For effectors where all CAR positivity percentages (for all 4 dilutions) are less than Min, then the one with the highest positivity percentage is chosen; if all are higher than the Max, then the one with the lowest positivity percentage is chosen; if two or more of them are between the Min and the Max, then the one closest to the Goal is chosen; and if only one of them is between the Min and the Max, then that one is chosen (Table 2).

TABLE 2
Example Selection Criteria

| Selection | A. | Minimum CAR positivity (“Min”) |
|---|---|---|
| cutoff values | B. | Goal CAR positivity (“Goal”) |
| | C. | Maximum CAR positivity (“Max”) |
| Input values | CAR positivity percentage (Pos %) at each dilution | |
| | level | |
| Selection | i. | if all Pos % > Max |
| Criteria | | => then select CAR-T with lowest Pos % |
| | ii. | if all Pos % < Min |
| | | => then select CAR-T with highest Pos % |
| | iii. | if at least two Pos % between Min and Max |
| | | => then select CAR-T with Pos % closest to |
| | | Goal |
| | iv. | if only one Pos % between Min and Max |
| | | => then select this one |

Upon such selections, the hit-picked CAR-T samples were further subjected to expansion and cytotoxicity testing. It was observed that, without such selection, the CAR-T drop out rates were about 60%. With the selection, the final drop out rates were about 14%, amounting to substantial improvements.

While a number of embodiments have been described, it is apparent that the disclosure and examples may provide other embodiments that utilize or are encompassed by the compositions and methods described herein. Therefore, it will be appreciated that the scope of is to be defined by that which may be understood from the disclosure and the appended claims rather than by the embodiments that have been represented by way of example.

1. A method for identifying T cells for expressing proteins encoded in polynucleotide vectors, comprising:
adding, to each of a plurality of T cell samples, one of a plurality (N) of polynucleotide vectors at one of a plurality (M) of amounts, each encoding a protein;
incubating each sample to allow the T cells to be transduced or transfected with the vector and express the protein;
measuring, in each sample, an expression level of the protein;
identifying, for each vector, a selected sample with one of the M amounts of the vector added, wherein the identification is at least in part based on the expression level; and
culturing each of the selected samples, thereby producing T cells expressing the proteins, wherein:
N is at least 2;
M is at least 2; and
for each vector, the difference between the highest amount and the lowest amount within the M amounts is at least 10 fold.

2. The method of claim 1, wherein each of the proteins is a chimeric antigen receptor (CAR) or a T cell receptor (TCR).

3. The method of claim 1, wherein the T cells are primary T cells.

4. The method of claim 1, wherein each of the polynucleotide vectors is a viral or retroviral vector.

5. (canceled)

6. The method of claim 1, wherein the expression level is a protein positivity level defined as a proportion of T cells of total live T cells in the sample that express the protein.

7. The method of claim 6, wherein identification of the selected samples comprises comparing the expression levels from each of the M amounts to predetermined expression level cutoff values, and wherein the predetermined expression level cutoff values comprise a maximum cutoff, a minimum cutoff and a goal cutoff.

8. (canceled)

9. The method of claim 7, wherein, for each vector:
(a) if the protein positivity levels of all M amounts are greater than the maximum cutoff, then the sample with the lowest protein positivity level is identified as the selected sample;
(b) if the protein positivity levels of all M amounts are lower than the minimum cutoff, then the sample with the highest protein positivity level is identified as the selected sample;
(c) if at least two of the protein positivity levels of all M amounts are between the maximum cutoff and the minimum cutoff, then the sample with a protein positivity level closest to the goal cutoff is identified as the selected sample; or
(d) if only one of the protein positivity levels of all M amounts is between the maximum cutoff and the minimum cutoff, then the sample having the protein positivity level between the maximum cutoff and the minimum cutoff is identified as the selected sample.

10. The method of claim 1, wherein M is at least 3.

11. (canceled)

12. The method of claim 1, wherein, for each vector, the difference between the highest amount and the lowest amount within the M amounts is at least 20 fold.

13. (canceled)

14. The method of claim 1, wherein all samples not identified as selected samples are not cultured together with the selected samples.

15. The method of claim 1, wherein the T cells are selected from a plurality of donors by a method comprising:
contacting T cells from each donor with a target tumor cell and measuring a first cytotoxicity level;
contacting T cells from the same donor, which have been engineered to express a reference CAR targeting a tumor marker associated with the target tumor cell, with the target tumor cell and measuring a second cytotoxicity level; and
selecting T cells, among the plurality of donors, having the highest difference between the second cytotoxicity level and the first cytotoxicity level.

16. The method of claim 15, wherein the highest difference is the highest fold difference.

17. The method of claim 1, wherein N is at least 8.

18. (canceled)

19. The method of claim 1, further comprising activating the T cells with an anti-CD3 and/or anti-CD28 antibody.

20. The method of claim 1, further comprising normalizing the number of T cells among the T cell samples.

21. The method of claim 1, wherein the M amounts of the vectors are made by dilutions from an initial amount.

22. The method of claim 21, wherein the initial amounts are normalized among the vectors.

23. The method of claim 21, wherein the initial amounts are not normalized among the vectors.

24. The method of claim 1, further comprising measuring the cytotoxicity of each cultured sample.
